(19) United States
(12) Patent Application Publication
(10) Pub. No.: US 2026/0070868 A1
(43) Pub. Date: Mar. 12, 2026

(54) PROCESSES FOR RECOVERING DIALKYL TEREPHTHALATES FROM POLYESTER COMPOSITIONS

(71) Applicant: Eastman Chemical Company, Kingsport, TN (US)

(72) Inventors: Pinguan Zheng, Johnson City, TN (US); Hannah Grace Harris, Kingsport, TN (US); Matthew F. Wickes, Indianapolis, IN (US); William Christopher Ketchie, Kingsport, TN (US)

(73) Assignee: Eastman Chemical Company, Kingsport, TN (US)

(21) Appl. No.: 18/703,825

(22) PCT Filed: Oct. 25, 2022
(86) PCT No.: PCT/US2022/047644

§ 371 Date: Apr. 23, 2024

Related U.S. Application Data
(60) Provisional application No. 63/262,977, filed on Oct. 25, 2021.

(51) Int. Cl. C07C 67/28; C07C 67/56

(52) U.S. Cl. CPC C07C 67/28; C07C 67/56

(57) ABSTRACT

Processes for recovering dialkyl terephthalates. The processes can include exposing a polyester composition to a first composition including one or more glycols and methanol under depolymerization conditions thereby providing one or more depolymerization products. The one or more depolymerization products can be exposed to an alcoholysis process to recover dialkyl terephthalate. Optionally, the one or more glycols and/or methanol can be recovered at the end of the alcoholysis process and re-used in subsequent processes for recovering dialkyl terephthalates.

### FIELD OF THE INVENTION

The present disclosure relates to processes for recycling polyester compositions. More particularly, the present disclosure relates to recovering dialkyl terephthalates from polyester compositions.

### BACKGROUND OF THE INVENTION

Certain conventional systems may utilize glycolysis and/or methanolysis processes in an attempt to recycle polyesters. However, certain conventional glycolysis and/or methanolysis processes may require a substantial amount of resources and energy in order to arrive at suitable products for use in subsequent production processes, e.g., production processes to generate recycled polyesters or other compositions.

### BRIEF SUMMARY OF THE INVENTION

In one aspect, a process for recovering one or more dialkyl terephthalates from a polyester composition is provided. The process includes exposing a first polyester composition to a first composition including one or more glycols and methanol in a first reaction vessel under depolymerization conditions to provide a first mixture, the first mixture comprising one or more first depolymerization products. The depolymerization conditions comprise a temperature of from 150° C. to 260° C. and a pressure of 10 atm to 70 atm. The process also includes cooling at least a portion of the first mixture to a temperature of 150° C. or below; and exposing the at least a portion of the first mixture to an alcohol composition and an alcoholysis catalyst under conditions including a temperature of from 23° C. to 90° C., and a pressure of from 1 atm to 2 atm for 0.5 hours to 5 hours to provide a second mixture. The second mixture including one or more dialkyl terephthalates. The process can also include separating at least a portion of the one or more dialkyl terephthalates by solid-liquid separation to provide a dialkyl terephthalate solid component and a filtrate.

### BRIEF DESCRIPTION OF THE DRAWING

FIG. 1 is an example system for recovering one or more dialkyl terephthalates from a polyester composition, in accordance with aspects of the present disclosure.

FIG. 2 is a graph of methyl ester to ethylene glycol ratio plotted against the ratio of methanol and ethylene glycol in the feed based on data provided in the Examples.

FIG. 3 is a graph of gas chromatography of a dimethyl terephthalate (DMT) product from Example 1.

### DETAILED DESCRIPTION OF THE INVENTION

### Overview

The present disclosure may be understood more readily by reference to the following detailed description of certain aspects of the disclosure and working examples. In according with the purpose(s) of this disclosure, certain aspects of the disclosure are described in the Brief Summary of the Invention and are further described herein below. Also, other aspects of the disclosure are described herein.

Aspects herein are directed to processes for recovering one or more dialkyl terephthalates from polyester compositions. As described herein, an example process can include exposing a polyester composition to one or more glycols and methanol under depolymerization conditions to generate one or more depolymerization products, which are then exposed to an alcoholysis process for recovery of dialkyl terephthalate.

As discussed above, certain conventional glycolysis and/or methanolysis processes may require a substantial amount of resources and energy in order to arrive at suitable products for use in subsequent production processes, e.g., production processes to generate recycled polyesters or other compositions.

The processes and systems disclosed herein can alleviate one or more of the above problems. For instance, in certain aspects, the processes disclosed herein can include exposing a polyester composition to depolymerization conditions with one or more glycols and methanol to provide one or more depolymerization products. In various aspects, the one or more depolymerization products can include monomers, oligomers, or a combination thereof. In aspects, the one or more depolymerization products can be exposed to alcoholysis conditions resulting in a dialkyl terephthalate product of high yield and purity. As discussed herein, the alcoholysis conditions include a temperature that is reduced compared to certain conventional systems, which reduces the overall energy and resources required.

### Polyester Compositions

As discussed above, the processes described herein relate to recovering one or more dialkyl terephthalates from a polyester composition. The term “polyester” can refer to a synthetic polymer prepared by the reaction of one or more difunctional carboxylic acids and/or multifunctional carboxylic acids with one or more difunctional hydroxyl compounds and/or multifunctional hydroxyl compounds. The difunctional carboxylic acid can be a dicarboxylic acid and the difunctional hydroxyl compound can be a dihydric alcohol such as, for example, glycols. Furthermore, as used herein, the term “diacid” or “dicarboxylic acid” includes multifunctional acids, such as branching agents. The term “glycol” or “diol” as used herein, includes, but is not limited to, diols, glycols, and/or multifunctional hydroxyl compounds. The dicarboxylic acid residues may be derived from a dicarboxylic acid monomer or its associated acid halides, esters, salts, anhydrides, or mixtures thereof. As used herein, therefore, the term dicarboxylic acid is intended to include dicarboxylic acids and any derivative of a dicarboxylic acid, including its associated acid halides, esters, halfesters, salts, half-salts, anhydrides, mixed anhydrides, or mixtures thereof, useful in a reaction process with a diol to make polyester. It should be understood, that the term “polyester” as used herein also refers to copolyesters.

As used herein, the term “residue(s)” refers to the monomer unit or repeating unit in a polymer, oligomer, or dimer. For example, a polymer can be made from the condensation of the following monomers: terephthalic acid (“TPA”) and cyclohexyl-1,4-dimethanol (“CHDM”). The condensation reaction results in the loss of water molecules. The residues in the resulting polymer are derived from either terephthalic acid or cyclohexyl-1,4-dimethanol. Below in Formula (I), a non-limiting example of a polyester is provided.

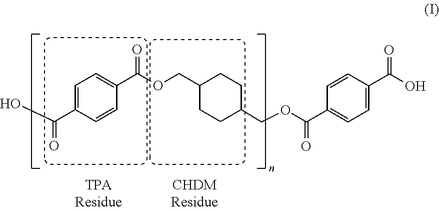

In aspects, the polyester composition exhibits an inherent viscosity of from about 0.1 dL/g to about 1.2 dL/g as determined according to ASTM D2857-70, about 0.2 dL/g to about 1.2 dL/g as determined according to ASTM D2857-70, about 0.3 dL/g to about 1.2 dL/g as determined according to ASTM D2857-70, or about 0.4 dL/g to about 1.2 dL/g as determined according to ASTM D2857-70.

In aspects, the polyester composition can include one or more polyesters. In various aspects, the one or more polyesters can include terephthalate polyesters. Terephthalate polyesters are polyesters that comprise residues of terephthalic acid or residues of any derivative of terephthalic acid, including its associated acid halides, esters, half-esters, salts, half-salts, anhydrides, mixed anhydrides, and/or mixtures thereof or residues thereof useful in a reaction process with a diol to make a copolyester In various aspects, the polyester composition can include polyethylene terephthalate (PET). In one or more aspects, the polyester composition can include glycol-modified PET. In certain aspects, the polyester composition can include polyethylene terephthalate (PET), 1,4-cyclohexanedimethanol (CHDM)-modified PET, isophthalic acid (IPA)-modified PET, diethylene glycol (DEG)-modified PET, glycol-modified PET, neopentyl glycol (NPG)-modified PET, propane diol (PDO)-modified PET, butanediol (BDO)-modified PET, heaxanediol (HDO)-modified PET, 2-methyl-2,4-pentanediol (MP diol)-modified PET, isosorbide-modified PET, polycyclohexylenedimethylene terephthalate (PCT), cyclohexanedimethanol (CHDM)-containing copolyester, isosorbide-containing copolyester, or a combination thereof.

In various aspects, the polyester composition can include CHDM. In one aspect, the polyester composition can include about 0 mole % to about 100 mole % CHDM, about 1 mole % to about 100 mole % CHDM, about 1 mole % to about 90 mole % CHDM, about 1 mole % to about 80 mole % CHDM, about 1 mole % to about 70% CHDM, about 1 mole % to about 60 mole % CHDM, about 1 mole % to about 50 mole % CHDM, about 1 mole % to about 40 mole % CHDM, about 1 mole % to about 35 mole % CHDM, about 1 mole % to about 30 mole % CHDM, about 1 mole % to about 25 mole % CHDM, about 1 mole % to about 20 mole % CHDM, about 1 mole % to about 10 mole % CHDM, or about 1 mole % to about 5 mole % CHDM. In aspects, the mole % of CHDM refers to the mole % of CHDM relative to all diol equivalents in the polyester composition. In various aspects, the polyester composition can include DEG. In aspects, the polyester composition can include about 0 mole % to about 100 mole % DEG, about 1 mole % to about 100 mole % DEG, about 1 mole % to about 90 mole % DEG, about 1 mole % to about 80 mole % DEG, about 1 mole % to about 70 mole % DEG, about 1 mole % to about 60 mole % DEG, about 1 mole % to about 50 mole % DEG, about 1 mole % to about 40 mole % DEG, about 1 mole % to about 35 mole % DEG, about 1 mole % to about 30 mole % DEG, about 1 mole % to about 20 mole % DEG, about 1 mole % to about 10 mole % DEG, about 1 mole % to about 5 mole % DEG, or about 1 mole % to about 3 mole % DEG. In aspects, the mole % of DEG refers to the mole % of DEG relative to all diol equivalents in the polyester composition. In aspects, the polyester composition can include isophthalic acid. In aspects, the polyester composition can include about 0 mole % to about 30 mole % isophthalic acid, about 1 mole % to about 30 mole % isophthalic acid, about 1 mole % to about 25 mole % isophthalic acid, about 1 mole % to about 20 mole % isophthalic acid, about 1 mole % to about 15 mole % isophthalic acid, about 1 mole % to about 10 mole % isophthalic acid, about 1 mole % to about 7.5 mole % isophthalic acid, about 1 mole % to about 5 mole % isophthalic acid, about 1 mole % to about 3 mole % isophthalic acid, about 10 mole % or less of isophthalic acid, about 7.5 mole % or less of isophthalic acid, about 5 mole % or less of isophthalic acid, or about 3 mole % or less of isophthalic acid. In aspects, the mole % of isophthalic acid refers to the mole % of isophthalic acid relative to all diacid equivalents in the polyester composition In certain aspects, the polyester composition can include about 0 mole % to about 100 mole % CHDM, about 0 mole % to about 100 mole % DEG, about 0 mole % to about 30 mole % isophthalic acid, or a combination thereof. In certain aspects, the polyester composition can include about 1 mole % to about 100 mole % CHDM, about 1 mole % to about 100 mole % DEG, about 1 mole % to about 30 mole % isophthalic acid, or a combination thereof. In various aspects, the polyester composition can include other glycols, e.g., other than those mentioned above. For instance, in aspects, the polyester composition can include, but is not limited to, neopentyl glycol (NPG), 2-methyl-2,4-pentanediol (MP diol), butanediol (BDO), propanediol (PDO), hexanediol (HDO), isosorbide, poly(tetramethylene ether) glycol (PTMG), poly(ethylene) glycol (PEG), or a combination thereof. In certain aspects, each of the NPG, MP diol, BDO, PDO, HDO, isosorbide, PTMG, and PEG can be present in the polyester composition in an amount of 0 mole % to about 100 mole %, about 1 mole % to about 100 mole %, about 1 mole % to about 90 mole %, about 1 mole % to about 80 mole %, about 1 mole % to about 70%, about 1 mole % to about 60 mole %, about 1 mole % to about 50 mole %, about 1 mole % to about 40 mole %, about 1 mole % to about 35 mole %, about 1 mole % to about 30 mole %, about 1 mole % to about 25 mole %, about 1 mole % to about 20 mole %, about 1 mole % to about 10 mole %, or about 1 mole % to about 5 mole %. In aspects, the mole % of each of NPG, MP diol, BDO, PDO, HDO, isosorbide, PTMG, and PEG refers to the mole % of each of NPG, MP diol, BDO, PDO, HDO, isosorbide, PTMG, and PEG, respectively, relative to all diol equivalents in the polyester composition. In various aspects, the polyester composition can include CHDM, DEG, NPG, MP diol, BDO, PDO, HDO, isosorbide, PTMG, PEG, isophthalic acid, or a combination thereof, where each component is present in any of the amounts for such components described in this paragraph.

In aspects, the polyester composition or the one or more polyesters present in the polyester composition can be recycled polyesters. In various aspects, the recycled polyester(s) can include material that was recovered as manufacturing scrap, industrial waste, post-consumer waste, or a combination thereof. In aspects, the recycled polyester(s) can be prior-used products that have been used and/or discarded. In aspects, the polyester composition and/or recycled polyester(s) can come from various sources and/or in various forms, including but not limited to textiles, carpet, thermoformed materials, bottles, pellets, and film. In one or more aspects, the polyester composition can include renewal polyesters, e.g., polyesters formed from DMT recovered from a prior DMT recovery process, such as the processes described herein.

In various aspects, the polyester composition can include one or more foreign materials. In aspects, the one or more foreign materials may include, but are not limited to, polyesters other than polyethylene terephthalate, polyvinyl chloride (PVC), polyvinyl acetal, polyvinyl butyral (PVB), polyvinyl alcohol (PVOH), ethylene vinyl alcohol (EVOH), cotton, polyolefins, polyethylene, polypropylene, polystyrene, polycarbonate, Spandex, natural fibers, cellulose ester, polyacrylates, polymethacrylate, polyamides, nylon, poly(lactic acid), polydimethylsiloxane, polysilane, calcium carbonate, titanium dioxide, inorganic fillers, dyes, pigments, color toners, colorants, plasticizers, adhesives, flame retardants, metals, aluminum, and iron, or a combination thereof. In various aspects, the one or more foreign materials can be present in the polyester composition in an amount of from about 0.01 wt. % to about 50 wt. %, about 0.01 wt. % to about 40 wt. %, about 0.01 wt. % to about 30 wt. %, about 0.01 wt. % to about 20 wt. %, about 0.01 wt. % to about 15 wt. %, about 0.01 wt. % to about 10 wt. %, about 0.01 wt. % to about 7.5 wt. %, about 0.01 wt. % to about 5 wt. %, about 0.01 wt. % to about 2.5 wt. %, about 0.01 wt. % to about 1.0 wt. %, relative to the weight of the one or more polyesters in the polyester composition.

In aspects, the polyester composition can be in solid form, liquid form, molten form, or in a solution. In certain aspects, the solution can include a polyester composition pre-dissolved in a solvent, e.g., DMT, EG, DEG, TEG, or a combination thereof.

### Optional Pre-treatment of the Polyester Composition

In certain aspects, an optional treatment of the polyester composition, prior to glycolysis and/or methanolysis, can be performed. In various aspects, the optional pretreatment can include any type of treatment that aids in removing a portion of any foreign materials from the polyester composition and/or that aids in recovering one or more polyesters from a mixed feedstock, e.g., a feedstock comprising the foreign materials discussed above. For instance, in one aspect, the optional pretreatment can include exposing to the polyester composition to one or more solvents, in an effort to selectively dissolve the polyester in the polyester composition (or at least a portion of the foreign materials in the polyester composition) to allow for separation between at least a portion of the foreign materials and the one or more polyesters in the polyester composition. As one example aspect, the optional pretreatment can include exposing the polyester composition to one or more solvents, e.g., one or more solvents that can cause dissolution of the polyester in the polyester composition. For instance, the one or more solvents can include but are not limited to 4-methylcyclohexanemethanol (MCHM), ethylene glycol (EG), diethylene glycol (DEG), triethylene glycol (TEG), 1,4-cyclohexanedimethanol (CHDM), poly(ethylene glycol) (PEG), neopentyl glycol (NPG), propane diol (PDO), butanediol (BDO), 2-methyl-2,4-pentanediol (MP diol), poly(tetramethylene ether)glycol (PTMG), dibutyl terephthalate (DBT), dioctyl terephthalate (DOTP), ethylene carbonate (EC), dimethyl carbonate (DMC), dimethyl sulfoxide (DMSO), dimethylformamide (DMF), or combinations thereof. In the same or alternative aspects, the polyester composition can be exposed to the one or more solvents at specific temperatures to effectuate dissolution of one or more components. In various aspects, a pretreatment process can include one or more dissolution and separation steps using various solvents and/or temperatures to achieve a desired level of foreign materials removal and/or purity level of PET. For instance, in one aspect, a dissolution and separation can be utilized using one solvent at a specific temperature, e.g., to remove one or more foreign materials, followed by a subsequent dissolution and separation of the polyester fraction using another solvent at a specific temperature, e.g., to remove one or more other foreign materials. The dissolution and/or separation(s) in this optional pretreatment step can utilize any suitable systems, reactors, vessels, and/or separation techniques to achieve a desired pretreated polyester composition.

Glycolysis and/or Depolymerization of the Polyester Composition

As discussed above, in various aspects, the processes disclosed herein can include exposing a polyester composition to depolymerization conditions to depolymerize at least a portion of the one or more polyesters into one or more depolymerization products. In various aspects, the one or more depolymerization products can include monomers, oligomers, or a combination thereof. In certain aspects, the oligomers can exhibit a degree of polymerization from 2 to 10, 2 to 8, 2 to 6, or 2 to 4. In aspects, the one or more polyesters may be depolymerized into one or more depolymerization products that can include monomers and terephthalate oligomers having a degree of polymerization from 2 to 10, 2 to 8, 2 to 6, or 2 to 4. In aspects, liquid chromatography can be utilized to discern the degree of polymerization of an oligomer, and/or gel permeation chromatography can be utilized to discern the molecular weight of the oligomers.

In aspects, the term degree of polymerization (DP) can refer to the number of residues in the oligomer. As used herein, the degree of polymerization (DP) refers to the number of difunctional carboxylic acid residues and/or multifunctional carboxylic acid residues in the oligomer. For instance, in one example aspect, a DP of one, would refer to a residue that includes one terephthalic acid residue or one isophthalic acid residue. In such an example aspect, a DP of one can also be referred to as a monomer. A non-limiting example of a DP of one is provided below in formula (II).

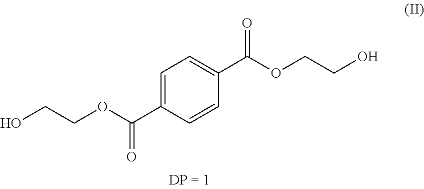 [SMILES: O=C(OCCO)c1ccc(C(=O)OCCO)cc1]

Formulas (III)-(V) below show non-limiting examples of oligomers having a DP of two, three, and n, respectively, in aspects.

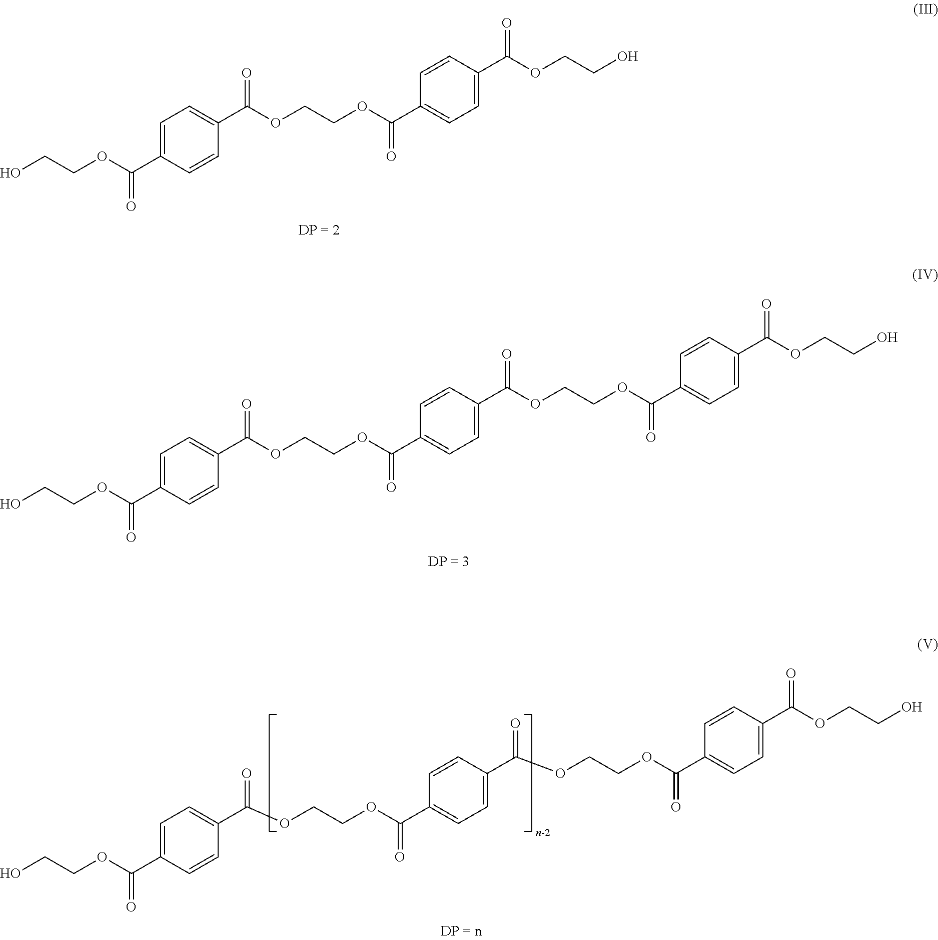

In aspects, this depolymerization can occur via a glycolysis process. Generally, in aspects, the glycolysis process disclosed herein can include exposing a polyester composition to one or more glycols and methanol, where the glycols and/or methanol react with the polyester, optionally in the presence of a trans-esterification catalyst, forming a mixture of bis(hydroxyethyl) terephthalate (BHET) or other terephthalate residue depending upon the glycol used (e.g., bis(2-hydroxydiethylene terephthalate) (BHDET) when DEG is utilized) and low molecular weight terephthalate oligomers. Some representative examples of glycolysis methods are disclosed in U.S. Pat. Nos. 3,257,335; 3,907,868; 6,706,843; 7,462,649, and are incorporated by reference herein.

In one aspect of a glycolysis process, one or more polyesters, e.g., one or more recycled polyesters, and one or more glycols and methanol can be fed into a glycolysis reactor where the one or more recycled polyesters are dissolved and depolymerized under depolymerization conditions.

In aspects, any amount of the one or more glycols suitable for use in a glycolysis process can be utilized. In various aspects, the weight ratio of the one or more glycols relative to the amount of the polyester composition can be of from 9:1 to 1:9, 8:1 to 1:9, 7:1 to 1:9, 6:1 to 1:9, 5:1 to 1:9, 4:1 to 1:9, 3:1 to 1:9, 2:9 to 1:9, 9:1 to 1:8, 9:1 to 1:7, 9:1 to 1:6, 9:1 to 1:5, 9:1 to 1:4, 9:1 to 1:3, or 9:1 to 1:2. In the same or alternative aspects, any amount of methanol suitable for use in the depolymerization or glycolysis process can be utilized. In certain aspects, a weight ratio of the amount of one or more glycols relative to the amount of methanol can be of from 1:20 to 100:1, 1:10 to 100:1, 1:5 to 100:1, 1:1 to 100:1, 1:20 to 80:1, 1:20 to 60:1, 1:20 to 50:1, 1:20 to 40:1, 1:20 to 30:1, 1:20 to 20:1, or 1:20 to 10:1.

In certain aspects, the one or more glycols can include any glycol suitable for use in a glycolysis process. As used herein, the term “glycol” refers to aliphatic, alicyclic, and aralkyl glycols. Exemplary glycols include ethylene glycol, 1,2-propandiol (also known propylene glycol), 1,3-propanediol, 1,4-butanediol, 1,5-pentanediol, 1,6-hexanediol, 2,2-dimethyl-1,3-propanediol, 1,2-cyclohexane dimethanol, 1,3-cyclohexane dimethanol, 1,4-cyclohexane dimethanol, 2,2,4,4-tetramethyl-1,3-cyclobutanediol, isosorbide, p-xylylenediol, and the like. These glycols may also contain ether linkages, such as is the case in, for example, diethylene glycol, triethylene glycol, and tetraethylene glycol. Additional embodiments of glycols include higher molecular weight homologs, known as polyethylene glycols, such as those produced by Dow Chemical Company under the Carbowax™ tradename. In one embodiment, the polyethylene glycol (PEG) has a molecular weight of from greater than 200 to about 10,000 Daltons (Mₙ). These glycols also include higher order alkyl analogs, such as dipropylene glycol, dibutylene glycol, and the like. Similarly, further glycols include higher order polyalkylene ether diols, such as polypropylene glycol and polytetramethylene glycol with molecular weights ranging from about 200 to about 10,000 Daltons (Mₙ) (also referred to as g/mole). In one aspect, the glycol can be chosen from aliphatic, alicyclic, and aralkyl glycols. In various aspects, the glycol can be chosen from ethylene glycol; 1,2-propandiol; 1,3-propanediol; 1,4-butanediol; 1,5-pentanediol; 1,6-hexanediol; 2,2-dimethyl-1,3-propanediol; 1,2-cyclohexane dimethanol; 1,3-cyclohexane dimethanol; 1,4-cyclohexane dimethanol; 2,2,4,4-tetramethyl-1,3-cyclobutanediol; isosorbide; p-xylylenediol; diethylene glycol; triethylene glycol; tetraethylene glycol; polyethylene glycols; dipropylene glycol; dibutylene glycol; polyalkylene ether diols chosen from polypropylene glycol and polytetramethylene glycol.

In certain aspects, the one or more glycols can include diethylene glycol (DEG), triethylene glycol (TEG), 1,4-cyclohexanedimethanol (CHDM), poly(ethylene glycol) (PEG), neopentyl glycol (NPG), propane diol (PDO), butanediol (BDO), 2-methyl-2,4-pentanediol (MP diol), poly(tetramethylene ether)glycol (PTMG), or a combination thereof; and, optionally, ii)ethylene glycol (EG). In such an aspect, the weight ratio of the glycols of group i) to EG can be of from 100:0 to 1:99. In certain aspects, the one or more glycols can include about 0 wt. % to about 100 wt. % DEG, or about 1 wt. % to about 100 wt. % DEG, relative to the total weight of the one or more glycols. In certain aspects, the one or more glycols can include about 0 wt. % to about 100 wt. % TEG, or about 1 wt. % to about 100 wt. % TEG, relative to the total weight of the one or more glycols. In aspects, the one or more glycols can include about 0 wt. % to about 50 wt. % CHDM, or about 1 wt. % to about 50 wt. % CHDM, relative to the total weight of the one or more glycols. In various aspects, the one or more glycols can include 0 wt. % to about 100 wt. % PEG, or about 1 wt. % to about 100 wt. % PEG, relative to the total weight of the one or more glycols. In various aspects, the one or more glycols can include 0 wt. % to about 100 wt. % NPG, or about 1 wt. % to about 100 wt. % NPG, relative to the total weight of the one or more glycols. In various aspects, the one or more glycols can include 0 wt. % to about 100 wt. % PDO, or about 1 wt. % to about 100 wt. % PDO, relative to the total weight of the one or more glycols. In various aspects, the one or more glycols can include 0 wt. % to about 100 wt. % BDO, or about 1 wt. % to about 100 wt. % BDO, relative to the total weight of the one or more glycols. In various aspects, the one or more glycols can include 0 wt. % to about 100 wt. % MP diol, or about 1 wt. % to about 100 wt. % MP diol, relative to the total weight of the one or more glycols. In various aspects, the one or more glycols can include 0 wt. % to about 100 wt. % PTMG, or about 1 wt. % to about 100 wt. % PTMG, relative to the total weight of the one or more glycols. In certain aspects, the one or more glycols can include about 0 wt. % to about 100 wt. % EG, or about 1 wt. % to about 100 wt. % EG, relative to the total weight of the one or more glycols. In one aspect, the one or more glycols can include 0 wt. % to about 100 wt. % EG, 0 wt. % to about 100 wt. % DEG, 0 wt. % to about 100 wt. % TEG, 0 wt. % to about 100 wt. % PEG, 0 wt. % to about 100 wt. % NPG, 0 wt. % to about 100 wt. % PDO, 0 wt. % to about 100 wt. % BDO, 0 wt. % to about 100 wt. % MP diol, 0 wt. % to about 100 wt. % PTMG, and 0 wt. % to about 50 wt. % CHDM, relative to the total weight of the one or more glycols. In certain aspects, as discussed in detail below, the one or more glycols can be recycle glycols that were recovered from a prior glycolysis and/or methanolysis process for recovery of one or more dialkyl terephthalates, as disclosed herein.

In various aspects, as discussed above, the glycolysis process can include one or more catalysts, e.g., trans-esterification catalysts. In certain aspects, the catalyst can be present in an amount of from 0.1 wt. % to 10 wt. %, relative to the weight of the polyester composition. In aspects, any suitable catalyst can be utilized. In one aspect, the catalyst can include a carbonate catalyst, for example, but not limited to: Li₂CO₃, K₂CO₃, Na₂CO₃, Cs₂CO₃, ZrCO₃, or a combination thereof. In one aspect, the catalyst can include a hydroxide catalyst, for example, but not limited to: LiOH, NaOH, KOH, tetrabutylammonium hydroxide (TBAH), or a combination thereof. In one aspect, the catalyst can include an alkoxide catalyst, for example, but not limited to: sodium methoxide (NaOMe), lithium methoxide (LiOMe), magnesium methoxide, potassium t-butoxide, ethylene glycol monosodium salt, ethylene glycol disodium salt, or a combination thereof. In one aspect, the catalyst can include tetra isopropyl titanate (TIPT), butyltin tris-2-ethylhexanoate (FASCAT 4102), 1,8-Diazabicyclo[5.4.0]undec-7-ene (DBU), zinc acetylacetonate hydrate (Zn(acac)₂), zinc acetate (Zn(OAc)₂), and manganese (II) acetate (Mₙ(OAc)₂)), or a combination thereof. In certain aspects, the catalyst can include LiOH, NaOH, KOH, tetra isopropyl titanate (TIPT), butyltin tris-2-ethylhexanoate (FASCAT 4102), ZrCO₃, 1,8-Diazabicyclo[5.4.0]undec-7-ene (DBU), sodium methoxide (NaOMe), lithium methoxide (LiOMe), and zinc acetylacetonate hydrate (Zn(acac)₂), or a combination thereof. In one aspect, the catalyst can include LiOH, NaOH, KOH, sodium methoxide (NaOMe), and lithium methoxide (LiOMe). In certain aspects, the catalyst can include Li₂CO₃, K₂CO₃, CaCO₃, Na₂CO₃, Cs₂CO₃, ZrCO₃, LiOH, NaOH, KOH, tetrabutylammonium hydroxide (TBAH), sodium methoxide (NaOMe), lithium methoxide (LiOMe), magnesium methoxide (Mg(OMe)₂, potassium t-butoxide, ethylene glycol monosodium salt, ethylene glycol disodium salt, tetra isopropyl titanate (TIPT), butyltin tris-2-ethylhexanoate (FASCAT 4102), 1,8-Diazabicyclo[5.4.0]undec-7-ene (DBU), zinc acetylacetonate hydrate (Zn(acac)₂), zinc acetate (Zn(OAc)₂), manganese (II) acetate (Mₙ(OAc)₂), hydrotalcite, zeolite, lithium chloride, or a combination thereof.

The depolymerization conditions can include a temperature of from 120° C. to 260° C. and an absolute pressure of from 10 atmospheres (atm) (147 psig) to 70 atm (1029 psig), in an agitated reactor for 0.5 h to 10 h. In aspects, the increased pressure can aid in preventing the methanol from vaporizing at the temperatures utilized in the depolymerization conditions. One or a plurality of reactors may be used for the reaction of the polyester with the one or more glycols and methanol. For example, the reaction mixture can be continuously withdrawn from the first stage and introduced to a second stage maintained under the aforementioned pressure range, along with additional glycol(s) and/or methanol, wherein depolymerization continues to the desired degree of completion. In various aspects, any type of vessel, reactor, and/or reactor system can be utilized for the depolymerization or glycolysis of the polyester composition. In one aspect, a continuous stirred-tank reactor or vessel, a fixed bed reactor, or a melt extruder can be utilized. In the same or alternative aspects, the depolymerization or glycolysis of the polyester composition can be a batch or continuous process.

Upon exposure to the depolymerization conditions detailed above, the resulting mixture can optionally be allowed to cool to a temperature of about 150° C. or less, or of from about 50° C. to about 150° C. In aspects, the resulting mixture can be allowed to cool to the desired temperature in the glycolysis reaction vessel(s) or can be transferred to a different vessel for temperature reduction. The resulting mixture can include a solid component and a liquid component. In aspects, the liquid component includes the one or more depolymerization products, e.g., monomers and/or oligomers having a degree of polymerization of from 2 to 10, along with the one or more glycols, and may also include any additional soluble components from the polyester composition, one or more glycols, catalysts, or a combination thereof. In various aspects, the solid component can be the residual foreign materials and any other insoluble components of the polyester composition and may be considered a waste product to discard.

As discussed further below, the liquid component is further subjected to at least a methanolysis and/or alcoholysis process for the recovery of one or more dialkyl terephthalates. In various aspects, prior to the methanolysis process, the liquid component can be separated from the solid component. In aspects, the liquid component can be separated from the solid component using any system. In one aspect, the liquid component can be separated from the solid component while the resulting mixture is at a temperature of from about 50° C. to about 150° C. In such aspects, separating the liquid component from the solid component at temperatures of about 150° C. or less, e.g., at about 50° C. to about 150° C., can provide for a more efficient process and/or less resource intensive process than current conventional processes. In the same or alternative aspects, separating the liquid component from the solid component at temperatures of about 150° C. or less, e.g., at about 50° C. to about 150° C., can be beneficial as some impurities can be unstable at higher temperatures, e.g., temperatures above 150° C., which may adversely affect the processes, product yields, and/or product purities described herein.

In various aspects, the separation of the liquid component from the solid component can include a filtration process. In such an aspect, any suitable filtration process can be utilized that is capable of withstanding the increased filtration temperatures of from about 50° C. to about 150° C. In certain aspects, the solid component can be removed by centrifugation. In certain aspects, the solid can be removed by settling or sedimentation. In certain aspects, the solid component may have settled in the bottom of a vessel allowing for removal of the liquid component through a vessel conduit or valve appropriately positioned within the vessel. In one aspects, such a conduit and/or valve may include a filtration device to minimize the inclusion of solid component in downstream processes.

### Alcoholysis of the One or More Depolymerization Products

As discussed above, in aspects, the one or more depolymerization products produced in the glycolysis process described above can be subjected to an alcoholysis process.

Generally, in a typical alcoholysis process, a polyester is reacted with an alcohol, e.g., methanol, to produce a depolymerized mixture comprising oligomers, terephthalate monomers, e.g., dimethyl terephthalate (DMT), and one or more glycols. In other embodiments, other monomers such as, for example, CHDM, DEG, and dimethyl isophthalate (DMI), also may be produced, depending on the composition of the polyester. In one embodiment, during the alcoholysis process the terephthalate oligomers are reacted with methanol to produce a depolymerized polyester mixture comprising polyester oligomers, DMT, CHDM, and/or EG.

Some representative examples of the methanolysis of PET are described in U.S. Pat. Nos. 3,321,510; 3,776,945; 5,051,528; 5,298,530; 5,414,022; 5,432,203; 5,576,456; 6,262,294; which are incorporated herein by reference.

In aspects, the alcoholysis process can include exposing the liquid component and/or the one or more depolymerization products resulting from the glycolysis process to an alcohol composition under conditions resulting in one or more dialkyl terephthalates. As discussed above, in aspects, the one or more depolymerization products can be present in the liquid component resulting from the glycolysis process. In various aspects, as discussed above, prior to subjecting the one or more depolymerization products and/or the liquid component resulting from the glycolysis process to an alcoholysis process, the liquid component can be separated from the resulting mixture and/or from the resulting solid component of the glycolysis process. In certain aspects, after separating the liquid component from the solid component of the glycolysis process, the liquid component can be directly utilized in this alcoholysis process. In the same or alternative aspects, after separating the liquid component from the solid component of the glycolysis process, the liquid component is not subjected to any further processing, e.g., distillation and/or other separation processes, prior to being utilized in this alcoholysis process. Without being bound by any particular theory, it is believed that since the glycolysis process is performed using a lower amount of glycols compared to certain conventional processes (or a weight ratio of glycols relative to the amount of the polyester composition of from 3:1 to 1:9) allows for the resulting liquid component from the glycolysis process to be directly utilized in the alcoholysis process without requiring further processing, e.g., to concentrate the resulting one or more depolymerization products and/or remove a portion of the glycols.

The alcohol composition can include any suitable alcohol known in the art for use in an alcoholysis process to obtain a specific dialkyl terephthalate. In one aspect, the alcohol composition can be and/or can include methanol. In aspects, when methanol is utilized as the alcohol composition, DMT can be the resulting methanolysis product.

In certain aspects, the amount of the alcohol composition can be any amount that is in excess on a weight basis relative to the amount or weight of the polyester composition. In certain aspects, a weight ratio of the amount of the alcohol composition relative to the amount of the polyester composition can be from about 2:1 to about 10:1. In various aspects, a weight ratio of the amount of the alcohol composition relative to the amount of the polyester composition can be from about 2:1 to about 9:1, about 2:1 to about 8:1, about 2:1 to about 7:1, about 2:1 to about 6:1, or about 2:1 to about 5:1. In aspects, the amount of the polyester composition refers to the amount or weight of the polyester composition that is utilized in the above glycolysis process.

In aspects, the alcoholysis reaction can occur at a temperature of about 90° C. or less, about 80° C. or less, about 70° C. or less, about 60° C. or less, about 50° C. or less, about 40° C. or less, or about 30° C. or less. In the same or alternative aspects, the alcoholysis reaction can occur at a temperature of from about 20° C. to about 90° C., about 20° C. to about 80° C., about 20° C. to about 70° C., about 20° C. to about 60° C., about 20° C. to about 50° C., about 20° C. to about 40° C., or about 20° C. to about 30° C. In certain aspects, the alcoholysis reaction can occur at a temperature of from about 25° C. to about 90° C., about 25° C. to about 80° C., about 25° C. to about 70° C., about 25° C. to about 60° C., about 25° C. to about 50° C., about 25° C. to about 40° C., or about 25° C. to about 30° C. In various aspects, without being bound by any particular theory, it is believed that, since in the processes disclosed herein, the polyester in the polyester composition has already undergone at least a partial depolymerization process, e.g., in the glycolysis step discussed above, that the methanolysis process can be performed at the temperatures described above, which are comparably reduced compared to certain other conventional processes. Additionally or alternatively, without being bound by any particular theory, it is believed that, since the one or more depolymerization products produced in the glycolysis process are separated from the waste or insoluble material prior to this alcoholysis process, the alcoholysis process can be conducted at the reduced temperatures described above.

In aspects, the alcoholysis process can be conducted in any suitable reactor and/or vessel. In aspects, the alcoholysis reactor can be in fluid communication with the reactor utilized in the glycolysis process described above. In certain aspects, the alcoholysis reactor is a different reactor than the vessel used for glycolysis. Alternatively, in various aspects, the alcoholysis process can be conducted in the same vessel as the glycolysis process and/or the filtration process discussed above. In certain aspects, the alcoholysis process can be conducted at ambient pressure, e.g., about 1 atm, or at a pressure of from about 1 atm to about 5 atm, or of from about 1 atm to about 3 atm, or of from about 1 atm to about 2 atm. In various aspects, the alcoholysis reaction can be conducted at a pressure above ambient pressure, e.g., more than 1 atm, or about 5 atm or less, about 3 atm or less, about 2 atm of less, when the alcoholysis reaction temperature is high for the process conditions disclosed herein, e.g., about 50° C. or more, about 60° C. or more, about 70° C. or more, about 80° C. or more, or about 90° C. or more.

In various aspects, an alcoholysis catalyst can be utilized in the alcoholysis process. In aspects, the alcoholysis catalyst can be present in an amount of from about 0.1 wt. % to about 20 wt. % relative to the weight of the polyester composition, or of from about 0.1 wt. % to about 10 wt. % relative to the weight of the polyester composition, or of from about 0.1 wt. % to about 5 wt. % relative to the weight of the polyester composition, or of from about 0.1 wt. % to about 2 wt. % relative to the weight of the polyester composition, or of from about 0.1 wt. % to about 1 wt. % relative to the weight of the polyester composition, or of from about 0.1 wt. % to about 0.5 wt. % relative to the weight of the polyester composition. In such aspects, the amount of the polyester composition refers to the amount or weight of the polyester composition that is utilized in the above glycolysis process. In various aspects, the alcoholysis catalyst amounts disclosed in this paragraph refer to the amount of alcoholysis catalyst present during the alcoholysis reaction. In various aspects, the alcoholysis catalyst amounts disclosed in this paragraph refer to the amount of alcoholysis catalysts that is added to one or more depolymerization products and the one or more alcohols to facilitate the alcoholysis reaction. In certain aspects, reduced or lower amounts of alcoholysis catalyst may be added to the one or more depolymerization products and the one or more alcohols to facilitate the alcoholysis reaction, such as an amount of from about 0.1 wt. % to about 10 wt. % relative to the weight of the polyester composition, or of from about 0.1 wt. % to about 5 wt. % relative to the weight of the polyester composition, or of from about 0.1 wt. % to about 2 wt. % relative to the weight of the polyester composition, or of from about 0.1 wt. % to about 1 wt. % relative to the weight of the polyester composition, or of from about 0.1 wt. % to about 0.5 wt. % relative to the weight of the polyester composition. In aspects, such a lower amount of alcoholysis catalysts may be added at least partly because alcoholysis catalyst is already present in the one or more depolymerization products and/or one or more alcohols. In such aspects, as discussed below, the alcohol and/or glycol may be recycled and re-used in subsequent glycolysis and alcoholysis process as disclosed herein, which may include at least a portion of alcoholysis catalyst from a prior alcoholysis and/or glycolysis process.

In various aspects, the alcoholysis catalyst can include a carbonate catalyst, for example, but not limited to: K₂CO₃, Na₂CO₃, Li₂CO₃, Cs₂CO₃; a hydroxide catalyst, for example, but not limited to: KOH, LiOH, NaOH; an alkoxide catalyst, for example, but not limited to NaOMe, Mg(OMe)₂, KOMe, KOt-Bu, ethylene glycol monosodium salt, ethylene glycol disodium salt, or a combination thereof. In certain aspects, the alcoholysis catalyst can include KOH, NaOH, LiOH, or a combination thereof. In certain aspects, the alcoholysis catalyst can include NaOMe, KOMe, Mg(OMe)₂, KOt-Bu, ethylene glycol monosodium salt, ethylene glycol disodium salt, or a combination thereof. In various aspects, the alcoholysis catalyst can be in solid form, a solution form in water, methanol, or ethylene glycol, or a combination of thereof. In certain aspects, the alcoholysis catalyst can be added to the one or more depolymerization products and the alcohol composition once the alcohol composition and the one or more depolymerization products reach the desired reaction temperature or temperature range disclosed above.

The one or more depolymerization products can be exposed to the alcohol composition and optionally the alcoholysis catalyst under the temperature and pressure conditions described above for a period of time to achieve the desired yield of the resulting dialkyl terephthalate. In certain aspects, the one or more depolymerization products can be exposed to the alcohol composition and optionally the alcoholysis catalyst under the temperature and pressure conditions described above for a period of time of from about 5 minutes to about 5 hours, or of from about 5 minutes to about 2 hours, or about 5 minutes to about 1 hour, or about 5 minutes to about 30 minutes, or about 5 minutes to about 15 minutes, or about 5 minutes to about 10 minutes.

In aspects, the alcoholysis process results in a mixture that includes one or more dialkyl terephthalates. In various aspects, the alcoholysis process results in mixture wherein the dialkyl terephthalate is an insoluble and/or solid component. In aspects, the liquid component of this mixture can include one or more glycols, methanol, the alcohol composition, or a combination thereof. In one aspect, the glycols and/or at least a portion of the methanol can be the glycols and/or methanol that were utilized in the glycolysis process and present with the one or more depolymerization products at the initiation of the alcoholysis process. In various aspects, the dialkyl terephthalate can be isolated from the mixture using any known separation technique, e.g., filtering, centrifugation, sedimentation, settling, or a combination of one or more separation techniques. In aspects, the filtering may include washing the solid component with additional alcohol composition or other solvent. The resulting liquid component can include the filtrate and wash. The resulting solid component can include about 90 wt. % or more dialkyl terephthalate, e.g., DMT, about 93 wt. % or more dialkyl terephthalate, e.g., DMT, or about 95 wt. % or more dialkyl terephthalate, e.g., DMT, relative to the weight of the solid component. In the same or alternative aspects, the dialkyl terephthalate, e.g., DMT, in the resulting solid component can be about 90% or more pure, about 93% or more pure, or about 95% or more pure. In various aspects, the solid component can also include dimethyl isophthalate (DMI). In such aspects, the DMI can be present in an amount of about 1000 ppm or less, or about 500 ppm or less, or of from about 1 ppm to about 1000 ppm, or of from about 1 ppm to about 500 ppm. In one or more aspects, the solid component can also include bisphenol A (BPA). In such aspects, the BPA can be present in an amount of about 1000 ppm or less, or about 500 ppm or less, or of from about 1 ppm to about 1000 ppm, or of from about 1 ppm to about 500 ppm.

The processes described herein, e.g., the glycolysis and/or alcoholysis processes are substantially mild compared to certain conventional processes, e.g., high temperature one-step glycolysis or methanolysis processes. For instance, certain conventional one-step processes may utilize a glycolysis process at temperatures of 240° C. or above in the presence of a Lewis acid catalyst, for instance, Zn(OAc)₂ or KOAc. Such harsh conditions can result in reduced EG yield from the depolymerization, as the EG is converted in various side reactions to various impurity compounds, including but not limited to: diethylene glycol (DEG), triethylene glycol (TEG), acetaldehyde, 1,1-dimethoxyethane, 1,2-dimethoxyethane, dioxane, 2-methoxyethanol, 1-methoxyethanol, and dimethyl ether. In aspects, the processes described herein are substantially milder than such conventional processes, and also result in less EG yield loss, e.g., from less side reactions converting EG into various impurities. In one aspect, the processes described herein result in about 5% or less yield loss of EG, about 2% or less yield loss of EG, or about 1% or less yield loss of EG, or about 0.5% or less yield loss of EG. In such aspects, the yield loss of EG is the percent of EG that is formed into impurities, e.g., DEG, relative to the combined amount of EG from the polyester composition feed and of the EG added in the glycolysis process. In the same or alternative aspects, the processes described herein result in minimal glycol impurities being produced. For instance, in one aspect, the processes described herein can result in the net generation of about 5 wt. % or less DEG, about 2 wt. % or less DEG, or about 1 wt. % or less DEG, or about 0.5 wt. % or less DEG, or of from about 0.01 wt. % to about 5 wt. % DEG, about 0.01 wt. % to about 2 wt. % DEG, or about 0.01 wt. % to about 1 wt. % DEG, or about 0.01 wt. % to about 0.5 wt. % DEG, or about 0.01 wt. % to about 0.2 wt. % DEG, when EG is used as the one or more glycols in the glycolysis process. In aspects, the processes described herein can result in the net generation of about 5 wt. % or less DEG and/or other impurity, about 2 wt. % or less DEG and/or other impurity, or about 1 wt. % or less DEG and/or other impurity, or about 0.5 wt. % or less DEG and/or other impurity, or of from about 0.01 wt. % to about 5 wt. % DEG and/or other 1 impurity, about 0.01 wt. % to about 2 wt. % DEG and/or other impurity, or about 0.01 wt. % to about 1 wt. % DEG and/or other impurity, or about 0.01 wt. % to about 0.5 wt. % DEG and/or other impurity, or about 0.01 wt. % to about 0.2 wt. % DEG and/or other impurity, when EG is used as the one or more glycols in the glycolysis process. In aspects, the net generation of DEG (or other impurity) is the weight percent of the amount of DEG or other impurity that is present over the amount of DEG or other impurity present in the polyester composition feed. In one aspect, the DEG being produced can be produced in the glycolysis process described herein and/or the alcoholysis process described herein. In certain aspects, the EG and/or any glycol impurities, such as DEG when using EG as the one or more glycols in the glycolysis process, can be present in the resulting liquid component from this alcoholysis step. In certain aspects, utilization of a Lewis base catalyst, e.g., a hydroxide-based or carbonate-based catalyst, in the glycolysis process may also facilitate or contribute to reduced EG degradation and/or a reduction of glycol impurities.

### Recycling Glycols, Methanol, and Alcohol Composition

As discussed above, in various aspects, the glycols and/or methanol utilized in the glycolysis process can be re-used in subsequent rounds of processes for recovery of one or more dialkyl terephthalates disclosed herein. At a high level, in aspects, the liquid component resulting from the alcoholysis process can be processed for re-use, e.g., for re-use in subsequent rounds of glycolysis and/or methanolysis of a subsequent polyester composition to recover one or more dialkyl terephthalates. In the same or alternative aspects, methanol can be recovered from the liquid component resulting from the alcoholysis process and can be re-used, e.g., in subsequent alcoholysis processes or can be used in the glycolysis or depolymerization processes described above. In such an aspect, the methanol can be recovered by exposing the liquid component to distillation conditions.

In aspects, as discussed above, the liquid component resulting from the alcoholysis process can include the glycols and/or methanol used in the glycolysis process, the alcohol composition, glycols generated in the glycolysis process, e.g., EG, DEG, and/or CHDM. In various aspects, this liquid component can be subjected to a separation process, e.g., to remove or separate at least a portion of the alcohol composition, for instance, methanol, or a mixture of methanol and ethylene glycol. In certain aspects, for removal of at least a portion of the alcohol composition, the liquid component can be exposed to distillation or short path distillation. In example non-limiting aspects, the distillation conditions may include exposing the liquid component to a temperature of about 260° C. or less, about 220° C. or less, about 200° C. or less, about 180° C. or less, about 160° C. or less, about 150° C. or less, about 130° C. or less, about 60° C. or more, about 70° C. or more, of from about 60° C. to about 220° C., of from about 70° C. to about 220° C., of from about 60° C. to about 180° C., or of from about 60° C. to about 160° C. In the same or alternative aspects, the distillation conditions can include a pressure of from about 1 Torr (133.3 Pa) to about 800 Torr (106,657 Pa), or about 30 Torr (3999 Pa) to about 500 Torr (66,661 Pa). In aspects, the liquid component can be exposed to the distillation conditions until all or a substantial portion of the alcohol composition has been removed, e.g., vaporized, from the liquid component. In certain aspects, at least a portion of the alcohol composition, e.g., the methanol, if present with the recycle glycols, may utilized during a subsequent glycolysis process. In certain aspects, the recovered alcohol from the liquid component, e.g., methanol, can be returned to the alcoholysis reaction vessel for use in subsequent alcoholysis processes and/or returned to a glycolysis reaction vessel for use in a glycolysis process as described above.

In aspects, the distillation of the liquid component can occur in any vessel or distillation system that is suitable for use in the processes and systems described herein. In one aspect, the distillation vessel can be in fluid communication with the alcoholysis reaction vessel and/or any component of the filtering process utilized subsequent to the alcoholysis, e.g., to isolate the dialkyl terephthalate solid or insoluble component. In the same or alternative aspects, the distillation vessel can be in fluid communication with the glycolysis vessel.

In various aspects, the distillation of the liquid component can cause the alcohol composition to vaporize leaving a pot residue. In various aspects, the pot residue includes the glycols and any other heavies, e.g., non-vaporizable compounds present in the liquid component. In aspects, the glycols in the pot residue can be referred to as recycle glycols and/or the glycols from a non-vaporizable portion of a continuous distillation process using the distillation conditions described herein can be referred to as recycle glycols. In one aspect, the recycle glycols can include ethylene glycol (EG), diethylene glycol (DEG), triethylene glycol (TEG), 1,4-cyclohexanedimethanol (CHDM), poly(ethylene glycol) (PEG), neopentyl glycol (NPG), propane diol (PDO), butanediol (BDO), 2-methyl-2,4-pentanediol (MP diol), poly(tetramethylene ether)glycol (PTMG), or a combination thereof.

In aspects, as discussed above, the recycle glycols can be utilized in a subsequent round of the process described herein to recover one or more dialkyl terephthalates from a polyester composition. Further, in aspects, the recycle glycols can be recycled again using the process described herein, after going through this subsequent round of dialkyl terephthalate recovery. In aspects, the recycle glycols can be recovered and re-used at least two, at least three, at least four, or at least five times. In certain aspects, when the recycle glycols are used in subsequent round(s) of dialkyl terephthalate recovery, addition of a catalyst in the subsequent glycolysis step(s) may be omitted, as the recycle glycols may include prior-used catalyst.

In aspects, when the recycles glycols are recovered and re-used, it has been unexpectedly found that the resulting dialkyl terephthalates recovered exhibit comparable purity to that of dialkyl terephthalates recovered using glycols that have not been recovered and re-used. This comparable purity of the dialkyl terephthalate is present, in aspects, after re-using recycle glycols at least two times, at least three times, at least four times, or at least five times resulting in a dialkyl terephthalate recovery having a purity of at least about 90%, at least about 93%, or at least about 95%.

### Example Systems

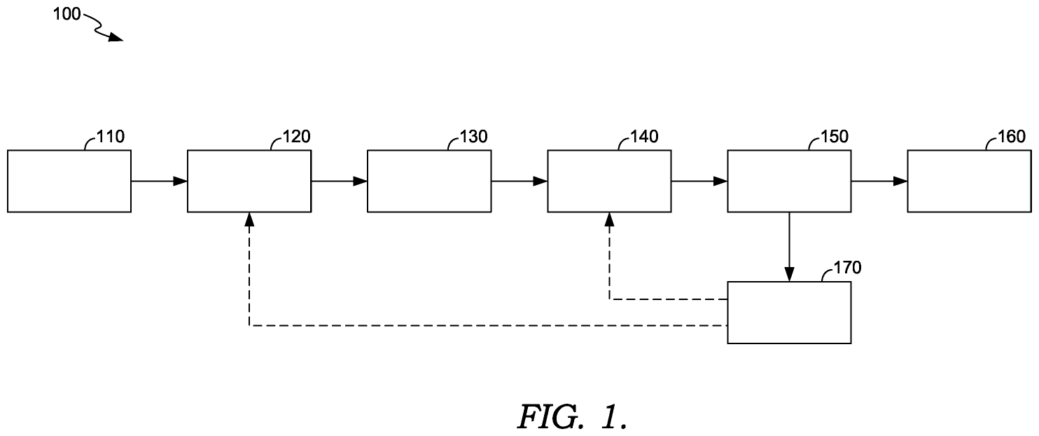

FIG. 1 schematically depicts one example system and/or process for recovering one or more dialkyl terephthalates from a polyester composition. The system 100 includes a source 110 of polyester composition, e.g., the polyester composition described above. In one example aspect, the polyester composition can undergo an optional pretreatment process, as discussed in detail above. In such an aspect, this optional pretreatment can occur prior to exposing the polyester composition to the depolymerization conditions in the vessel 120. The vessel 120 represents the glycolysis vessel, where the polyester composition is received and exposed to one or more glycols and methanol under depolymerization conditions, as discussed in detail above. In aspects, the vessel 120 can be in fluid communication with the source 110. In various aspects, as discussed above, the polyester composition, after exposure to the depolymerization conditions in the vessel 120, is converted into one or more depolymerization products. In various aspects, as discussed above, the one or more depolymerization products can include monomers and/or oligomers having a degree of polymerization of from 2 to 10, 2 to 8, 2 to 6, or 2 to 4. In aspects, the one or more depolymerization products are present in a mixture that includes a liquid component and a solid component, with the one or more depolymerization products in the liquid component. In aspects, as discussed above, this mixture is exposed to a solid-liquid separation device 130, e.g., a filtering system, where the liquid component, containing the one or more depolymerization products, is separated from the solid component. In various aspects, as discussed herein, the solid-liquid separation device 130 can be in fluid communication with the vessel 120 and/or with the vessel 140. In the aspect depicted in FIG. 1, the one or more depolymerization products and/or the liquid component can be exposed to alcoholysis conditions in a vessel 140. In aspects, the one or more depolymerization products and/or the liquid component can be directly utilized in this alcoholysis process. In such an aspect, the one or more depolymerization products and/or the liquid component may not be subjected to any further processing, e.g., distillation and/or other separation processes, prior to being utilized in this alcoholysis process. Alcoholysis conditions are discussed in detail above. In aspects, as discussed above, the alcoholysis of the one or more depolymerization products and/or the liquid component can result in a mixture that includes an insoluble or solid component that comprises the dialkyl terephthalate and a liquid component that comprises the alcohol composition, glycols, methanol, and potentially other soluble components as described herein. As discussed above, the resulting alcoholysis reaction mixture can be exposed to a solid-liquid separation device 150, e.g., a filtering system, to separate the solid component containing the recovered dialkyl terephthalate 160. In aspects, the solid-liquid separation device 150 can be in fluid communication with the vessel 140. In various aspects the liquid component resulting from the solid-liquid separation in the solid-liquid separation device 150 can include one or more alcohols, e.g., methanol, and/or one or more glycols. In such aspects, this liquid component can be exposed to one or more distillation or other separation processes in the system 170. At the system 170, one or more alcohols, e.g., methanol, may be separated from the liquid component as discussed above, and optionally returned to the vessel 140 for use in subsequent alcoholysis processes and/or returned to the vessel 120 for us in subsequent glycolysis processes. In the same or alternative aspects, at the system 170, one or more glycols can be recovered and returned to the vessel 120, alone or with recovered methanol as well, for subsequent depolymerization processes, as discussed above. Further, in various aspects, the system, 170 can selectively remove the EG from the liquid component for storage or purposes other than returning to the vessel 120. The system 170 can be any type of separation or distillation system that is suitable for selectively recovering one or more alcohols and/or one or more glycols from the liquid component. In certain aspects, the process described herein associated with the system 100 can be performed as a continuous process, a batch process, or a semi-continuous process. It is understood that the system 100 is just one example system and other configurations of system components are contemplated by the disclosure herein. For instance, one or more of the components of the system 100 may not be physically separated, or distinct, from one or more other components of the system 100. It is further understood that the system 100 is only schematically depicted in order to highlight aspects of the processes disclosed herein.

### Use of Recovered Dialkyl Terephthalates to Form Polyesters or Other Products

As discussed above, the processes disclosed herein can result in high purity dialkyl terephthalates, such as DMT. For instance, in certain aspects, the recovered DMT can be utilized to form one or more polyesters, including but not limited to PET and TMCD-containing polyesters. In certain aspects, the polyesters formed using recovered DMT can be termed renewal polyesters. In various aspects, the products formed using the recovered DMT may be indistinguishable from similar products formed from virgin DMT. In such aspects, any suitable process for forming the PET and TMCD-containing polyesters can be utilized, since the DMT is of sufficient purity.

In the same or alternative aspects, the recovered DMT can be utilized to form CHDM. In various aspects, the CHDM formed using recovered DMT may be indistinguishable from CHDM formed from virgin DMT, due to the high purity of the recovered DMT. In such aspects, the CHDM can be formed from the recovered DMT using any suitable process.

In various aspects, the recovered DMT can be utilized to form one or more plasticizers. In certain aspects, the plasticizers formed using recovered DMT can include dibutyl terephthalate (DBT) and/or dioctyl terephthalate (DOTP). In various aspects, the DBT and/or DOTP formed using recovered DMT may be indistinguishable from the DBT and/or DOTP, respectively, formed from virgin DMT, due to the high purity of the recovered DMT. In such aspects, the DBT and/or DOTP can be formed from the recovered DMT using any suitable process.

### EXAMPLES

### Materials

FDST-5 contains 100 mole % TPA, 93.0 mole % EG, 4.1 mole % CHDM, and 2.9 mole % DEG, and is available from Eastman. IV: 0.751 dL/g.

Ethylene glycol (EG), methanol, potassium carbonate, 50% sodium hydroxide aqueous solution, were obtained from commercial vendors. All chemicals and reagents were used as received, unless otherwise mentioned.

### Analytical Procedures

Gas Chromatography (GC) Analysis. GC analysis was performed on an Agilent model 7890B gas chromatograph equipped with a 7693A autosampler and two G4513A towers. The gas chromatograph (GC) was outfitted with two columns—a 60 m×0.32 mm×1.0 micron DB-1701™ (J&W 123-0763) and a 60 m×0.32×1 micron DB-1™ (J&W 123-1063)—and samples were injected simultaneously onto both columns. A shared oven temperature program was used, and sample components were detected by flame ionization detection (FID). Five-point calibrations were performed for components of interest. The gas chromatograph was interfaced to an EZChrom Elite Chromatography Data System.

Methanolysis product samples were prepared by adding a known volume of pyridine-based internal standard solution to a known mass of sample and then derivatizing with N,O-Bis(trimethylsilyl)trifluoroacetamide (BSTFA). BHET (bis(hydroxyethyl) terephthalate) GC wt %, MHT (4-(methoxycarbonyl) benzoic acid) GC wt. %, MHET (Methyl-2-hydroxyethyl terephthalate) GC wt. % are provided as a read-out from the GC process software.

DMT (dimethyl terephthalate) % yield was calculated as: (weight of final DMT)/(theoretical DMT weight)*100%.

DMT GC purity % was calculated as: (weight % of DMT in final product by GC)/(total wt. % by GC)*100%. Major impurities shown in GC include MeOH, water and EG. DMT purity for almost every example is greater than 99% if excluding MeOH, water and EG.

Inherent Viscosity Measurement. The Inherent viscosities (IV) of the particular polymer materials useful herein are determined according to ASTM D2857-70 procedure, in a Wagner Viscometer of Lab Glass, Inc., having a ½ mL capillary bulb, using a polymer concentration about 0.5% by weight in 60/40 by weight of phenol/tetrachloroethane. The procedure is carried out by heating the polymer/solvent system at 120° C. for 15 minutes, cooling the solution to 25° C. and measuring the time of flow at 25° C. The IV is calculated from the equation:

$${η}_{inh} = \frac{ln \frac{{t}_{s}}{{t}_{0}}}{C}$$

where: η: inherent viscosity at 25° C. at a polymer concentration of 0.5 g/100 mL of solvent; tS: sample flow time; t0: solvent-blank flow time; C: concentration of polymer in grams per 100 mL of solvent. The units of the inherent viscosity throughout this application are in the deciliters/gram.

Viscosity can be measured in tetrachloroethane/phenol (50/50, weight ratio) at 30° C. and calculated in accordance with the following equation:

$${η}_{inh} = \frac{ln ( {η}_{sp} )}{C}$$

wherein ηₛₚ is a specific viscosity and C is a concentration.

### Example 1: PET Depolymerization with a 1:1 wt. Ratio of EG and Methanol at 195° C., Followed by Methanolysis

In this Example 1, for the depolymerizatrion, a 100 mL autoclave was equipped with a mechanical stirrer, thermocouple, gas inlet, and vent. Pulverized FDST-5 (15.18 g), ethylene glycol (15.57 g), methanol (15.14 g), potassium carbonate (0.1533 g) was charged to the reactor. The vessel was pressurized to 150 psig using nitrogen and the mixture was heated to 195° C. Once internal temperature reached 195° C., the pressure was adjusted to 750 psig. The conditions were held for 3 hours. The crude mixture was allowed to cool to 25° C. and pressure was released. GC analysis showed that crude mixture contained 5.2% BHET, 14.4% MHET and 17.4% DMT.

For the methanolysis, crude product was diluted with MeOH (45.0 g) was added and heated to 50° C. NaOH (50 wt %, 93 uL) was added. The resulting mixture was held at 50° C. for 30 min before it was cooled down to ambient temperature and held for an additional 2 hours. After filtration and MeOH washes, the product was isolated as white solid (85.0% yield, 99% purity).

### Example 2: PET Depolymerization with a 1:1 wt. Ratio of EG and Methanol at 215° C.

A 100 mL autoclave was equipped with a mechanical stirrer, thermocouple, gas inlet, and vent. Pulverized FDST-5 (15.06 g), ethylene glycol (15.22 g), methanol (15.07 g), potassium carbonate (0.1554 g) was charged to the reactor. The vessel was pressurized to 150 psig using nitrogen and the mixture was heated to 215° C. Once internal temperature reached 215° C., the pressure was adjusted to 750 psig. The conditions were held for 3 hours. The crude mixture was allowed to cool to 25° C. and pressure was released. GC analysis showed that crude mixture contained 5.2% BHET, 14.9% MHET and 15.1% DMT.

### Example 3: PET Depolymerization with a 4:1 wt. Ratio of EG and Methanol at 195° C.

A 100 mL autoclave was equipped with a mechanical stirrer, thermocouple, gas inlet, and vent. Pulverized FDST-5 (15.01 g), ethylene glycol (24.48 g), methanol (6.07 g), potassium carbonate (0.1504 g) was charged to the reactor. The vessel was pressurized to 150 psig using nitrogen and the mixture was heated to 195° C. Once internal temperature reached 195° C., the pressure was adjusted to 750 psig. The conditions were held for 3 hours. The crude mixture was allowed to cool to 25° C. and pressure was released. GC analysis showed that crude mixture contained 16.2% BHET, 15.7% MHET and 3.8% DMT.

### Example 4: PET Depolymerization with a 1:0 wt. Ratio of EG and Methanol at 195° C.

A 500 mL RB (round bottom) flask was equipped with a mechanical stirrer, thermocouple, gas inlet, and vent. Pulverized FDST-5 (30 g), ethylene glycol (70 g), and potassium carbonate (1 wt. %) was charged to the reactor. The mixture was heated to 195° C. at ambient pressure. The conditions were held for 3 hours. The crude mixture was allowed to cool to 25° C. GC analysis showed that crude mixture contained 33.1% BHET, and no DMT.

### Example 5: PET Depolymerization with a 0:1 wt. Ratio of EG and Methanol at 195° C.

A 100 mL autoclave was equipped with a mechanical stirrer, thermocouple, gas inlet, and vent. Pulverized FDST-5 (15.01 g), methanol (approx. 30 g), potassium carbonate (0.1504 g) was charged to the reactor. The vessel was pressurized to 150 psig using nitrogen and the mixture was heated to 195° C. Once internal temperature reached 195° C., the pressure was adjusted to 750 psig. The conditions were held for 3 hours. The crude mixture was allowed to cool to 25° C. and pressure was released. GC analysis showed that crude mixture contained 1.2 wt. % 0.1 wt. % BHET, 3.7 wt. % MHET, and 39.9 wt. % DMT.

Tables 1 and 2 below summarize the results of the above Examples.

TABLE 1
Summary of Examples and GC Analysis of Product

| | MeOH/EG in | | MHT GC | BHET | MHET | Dimer | DMT GC |
|---|---|---|---|---|---|---|---|
| Example | the feed | T (° C.) | wt % | GC wt % | GC wt % | calculated wt % | wt % |
| Example 4 | 0/100 | 195 | 0 | 33.1 | 0 | 7.1 | 0 |
| Example 3 | 20/80 | 195 | 0.4 | 16.2 | 15.7 | 2.5 | 3.8 |
| Example 1 | 50/50 | 195 | 0.0 | 5.2 | 14.4 | 0.2 | 17.4 |
| Example 2 | 50/50 | 215 | 0.0 | 5.2 | 14.9 | 0.3 | 15.1 |
| Example 5 | 100/0 | 195 | 1.2 | 0.1 | 3.7 | 0 | 39.9 |

TABLE 2
Relative Amounts of Methyl ester to EG ratio (Me/EG)

| | Me/EG ratio | MeOH/EG mol ratio | Me/EG ester |
|---|---|---|---|
| Example | (mol) | excluding EG in PET | mol ratio |
| Example 4 | 0 | 0 | 0 |
| Example 3 | 0.40 | 0.48 | 0.6 |
| Example 1 | 1.46 | 1.94 | 2.1 |
| Example 2 | 1.46 | 1.94 | 2.0 |
| Example 5 | 12.00 | — | 29.4 |

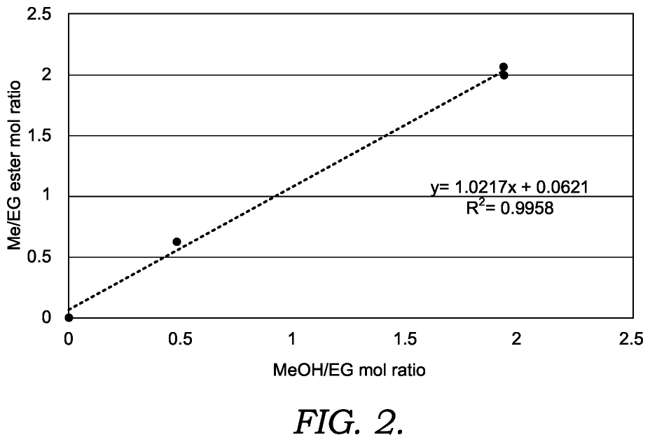

As shown in Table 1, BHET % and DMT % in the depolymerization product has strong correlation to the ratio of MeOH/EG. Further, MHET % stayed around 15% when using a different ratio of MeOH/EG, while the MHET level was significantly lower when using pure EG or MeOH. As shown in Table 2 and FIG. 2, the ratio of methyl ester/EG ester was plotted against the ratio of MeOH/EG in the feed. The plot suggested that methanol had a similar reactivity as EG as indicated by the slope of 1.02 and R² of 0.99.

### Example 6: DMT Product GC and NMR Analysis

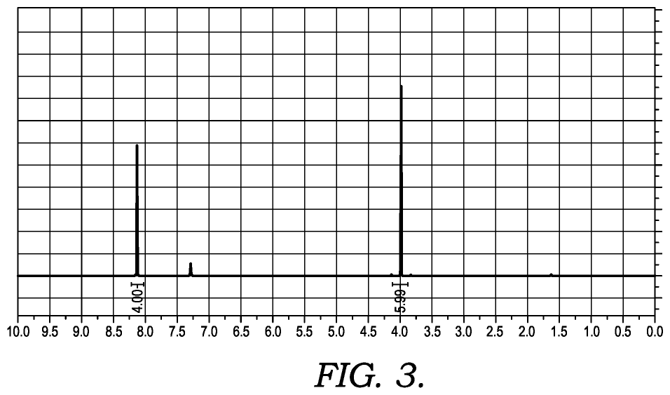

The crude depolymerization product of Example 1 was further subjected to the methanolysis condition at 50° C. DMT product was isolated in 85% yield in two steps. Both NMR and GC showed that the product has 99% purity. FIG. 3 shows a graph of the GC data.

The present disclosure can also be described in accordance with the following numbered clauses.
Clause 1. A process for recovering one or more dialkyl terephthalates from a polyester composition, comprising: exposing a first polyester composition to a first composition comprising one or more glycols and methanol in a first reaction vessel under depolymerization conditions to provide a first mixture, the first mixture comprising one or more first depolymerization products, and wherein the depolymerization conditions comprise a temperature of from 150° C. to 260° C. and a pressure of 10 atm to 70 atm; cooling at least a portion of the first mixture to a temperature of 150° C. or below; exposing the at least a portion of the first mixture to an alcohol composition and an alcoholysis catalyst under conditions including a temperature of from 23° C. to 90° C., and a pressure of from 1 atm to 2 atm for 0.5 hours to 5 hours to provide a second mixture, the second mixture comprising one or more dialkyl terephthalates; and separating at least a portion of the one or more dialkyl terephthalates by solid-liquid separation to provide a dialkyl terephthalate solid component and a filtrate. Clause 2. The process of clause 1, wherein at least a portion of the methanol was recovered from a prior process of recovering dialkyl terephthalate from a prior polyester composition.
Clause 3. The process of clause 1, wherein the filtrate comprises one or more glycols and methanol.
Clause 4. The process of clause 3, further comprising: exposing a second polyester composition to at least a portion of the filtrate in the first reaction vessel under the depolymerization conditions to produce one or more second depolymerization products.
Clause 5. The process of clauses 1-4, wherein a weight ratio of the amount of the one or more glycols relative to the amount of the methanol in the first reaction vessel is 1:20 to 100:1.
Clause 6. The process of clauses 1-5, wherein the cooling at least a portion of the first mixture to a temperature of 150° C. or below comprises, cooling the at least a portion of the first mixture to a temperature of from 50° C. to 150° C.
Clause 7. The process of clauses 1-6, further comprising: separating at least a portion of the one or more first depolymerization products in the first mixture from an insoluble component in the first mixture, wherein the separating occurs at a temperature of from 50° C. to 150° C.
Clause 8. The process of clauses 1-7, wherein, during the exposing a first polyester composition to a first composition comprising one or more glycols and methanol in a first reaction vessel under depolymerization conditions, a weight ratio of the one or more glycols to the first polyester composition is in a range of about 1:9 to about 9:1.
Clause 9. The process of clauses 1-8, wherein the one or more glycols comprises ethylene glycol (EG), diethylene glycol (DEG), triethylene glycol (TEG), 1,4-cyclohexanedimethanol (CHDM), poly(ethylene glycol) (PEG), neopentyl glycol (NPG), propane diol (PDO), butanediol (BDO), 2-methyl-2,4-pentanediol (MP diol), poly(tetramethylene ether)glycol (PTMG), or a combination thereof.
Clause 10. The process of clauses 1-9, wherein a weight ratio of the alcohol composition to the first polyester composition can be in a range of about 2:1 to about 10:1.
Clause 11. The process of clauses 1-10, wherein the first polyester composition comprises polyethylene terephthalate (PET), 1,4-cyclohexanedimethanol (CHDM)-modified PET, isophthalic acid (IPA)—modified PET, diethylene glycol (DEG)-modified PET, neopentyl glycol (NPG)-modified PET, propane diol (PDO)-modified PET, butanediol (BDO)-modified PET, heaxanediol (HDO)-modified PET, 2-methyl-2,4-pentanediol (MP diol)-modified PET, isosorbide-modified PET, poly(tetramethylene ether) glycol (PTMG)-modified PET, poly(ethylene glycol) (PEG)-modified PET, polycyclohexylenedimethylene terephthalate (PCT), cyclohexanedimethanol (CHDM)-containing copolyester, isosorbide-containing copolyester, or a combination thereof.
Clause 12. The process of clauses 1-11, wherein the first polyester composition contains 0 mole % to 100 mole % CHDM, 0 mole % to 100 mole % DEG, 0 mole % to 100 mole % NPG, 0 mole % to 100 mole % PDO, 0 mole % to 100 mole % BDO, 0 mole % to 100 mole % HDO, 0 mole % to 100 mole % MP diol, 0 mole % to 100 mole % isosorbide, 0 mole % to 100 mole % PTMG, 0 mole % to 100 mole % PEG, and 0 mole % to 30 mole % isophthalic acid, wherein the sum of diol equivalents in the one or more polyesters is about 100 mole %, and wherein the sum of diacid equivalents in the first polyester composition is about 100 mole %.
Clause 13. The process of clauses 1-12, wherein the polyester composition has an inherent viscosity of from about 0.1 dL/g to about 1.2 dL/g, as determined according to ASTM D2857-70.
Clause 14. The process of clauses 1-13, wherein one or more polyesters present in the first polyester composition are recycled polyesters.
Clause 15. The process of clauses 1-14, wherein the first polyester composition comprises one or more foreign materials, the one or more foreign materials comprise at least one member selected from the group consisting of polyesters other than polyethylene terephthalate, polyvinyl chloride (PVC), polyvinyl acetal, polyvinyl butyral (PVB), polyvinyl alcohol (PVOH), ethylene vinyl alcohol (EVOH), cotton, polyolefins, polyethylene, polypropylene, polystyrene, polycarbonate, Spandex, natural fibers, cellulose ester, polyacrylates, polymethacrylate, polyamides, nylon, poly(lactic acid), polydimethylsiloxane, polysilane, calcium carbonate, titanium dioxide, inorganic fillers, dyes, pigments, color toners, colorants, plasticizers, adhesives, flame retardants, metals, aluminum, and iron.
Clause 16. The process of clause 15, wherein the one or more foreign materials are present in the polyester composition in an amount of from 0.01 wt. % to 50 wt. %, relative to the weight of one or more polyesters in the polyester composition.
Clause 17. The process of clauses 1-16, wherein the dialkyl terephthalate solid component comprises dimethyl terephthalate (DMT), and wherein the DMT is at least 90% pure.
Clause 18. The process of clauses 1-17, wherein the dialkyl terephthalate solid component further comprises: dimethyl isophthalate (DMI) in an amount of 1000 ppm or less, or 500 ppm or less; bisphenol A (BPA) in an amount of 1000 ppm or less, or 500 ppm or less; or both.
Clause 19. The process of clauses 1-18, wherein the exposing a first polyester composition to a first composition comprising one or more glycols and methanol in a first reaction vessel under depolymerization conditions comprises further exposing the first polyester composition to one or more glycolysis catalysts, wherein the one or more glycolysis catalysts comprise a member selected from the group consisting of Li₂CO₃, K₂CO₃, CaCO₃, Na₂CO₃, Cs₂CO₃, ZrCO₃, LiOH, NaOH, KOH, tetrabutylammonium hydroxide (TBAH), sodium methoxide (NaOMe), lithium methoxide (LiOMe), magnesium methoxide (Mg(OMe)₂, potassium t-butoxide, ethylene glycol monosodium salt, ethylene glycol disodium salt, tetra isopropyl titanate (TIPT), butyltin tris-2-ethylhexanoate (FASCAT 4102), 1,8-Diazabicyclo[5.4.0]undec-7-ene (DBU), zinc acetylacetonate hydrate (Zn(acac)₂), zinc acetate (Zn(OAc)₂), manganese (II) acetate (Mₙ(OAc)₂), hydrotalcite, zeolite, and lithium chloride. Clause 20. The process of clause 19, wherein the one or more glycolysis catalysts comprise a member selected from the group consisting of LiOH, NaOH, KOH, tetra isopropyl titanate (TIPT), butyltin tris-2-ethylhexanoate (FASCAT 4102), ZrCO₃, 1,8-Diazabicyclo[5.4.0]undec-7-ene (DBU), sodium methoxide (NaOMe), lithium methoxide (LiOMe), zinc acetylacetonate hydrate (Zn(acac)₂), Cs₂CO₃, ethylene glycol sodium salt, and manganese (II) acetate (Mₙ(OAc)₂).
Clause 21. The process of clause 19, wherein the one or more glycolysis catalysts comprise a member selected from the group consisting of LiOH, NaOH, KOH, sodium methoxide (NaOMe), Cs₂CO₃, ethylene glycol sodium salt and lithium methoxide (LiOMe).
Clause 22. The process of clauses 15-21, further comprising: prior to the exposing the at least a portion of the first mixture to an alcohol composition and an alcoholysis catalyst, separating an insoluble component in the first mixture, wherein the insoluble component comprises the one or more foreign materials.
Clause 23. The process of clause 22, wherein the separating comprises filtration, centrifugation, settling, sedimentation, or a combination thereof.
Clause 24. The process of clauses 1-23, wherein the alcohol composition comprises methanol.
Clause 25. The process of clauses 1-24, wherein, the alcoholysis catalyst is present in an amount of from 0.1 wt. % to 20 wt. %, relative to the weight of the first polyester composition.
Clause 26. The process of clause 1-25, wherein the alcoholysis catalyst comprises K₂CO₃, Na₂CO₃, Li₂CO₃, Cs₂CO₃; KOH, LiOH, NaOH; NaOMe, Mg(OMe)₂, KOMe, KOt-Bu, ethylene glycol monosodium salt, ethylene glycol disodium salt, or a combination thereof.
Clause 27. The process of clause 26, wherein the alcoholysis catalyst comprises KOH, NaOH, NaOMe or a combination thereof.
Clause 28. The process of clauses 1-27, wherein the process is conducted as a batch process, a semi-continuous process, or a continuous process.
Clause 29. The process of clauses 1-28, wherein the one or more depolymerization products comprise monomers, oligomers, or a combination thereof.
Clause 30. The process of clause 29, wherein the one or more oligomers exhibit a degree of polymerization of from 2 to 10.

This disclosure has been described in detail with particular reference to specific aspects thereof, but it will be understood that variations and modifications can be made within the spirit and scope of this disclosure.

1. A process for recovering one or more dialkyl terephthalates from a polyester composition, comprising: exposing a first polyester composition to a first composition comprising one or more glycols and methanol in a first reaction vessel under depolymerization conditions to provide a first mixture, the first mixture comprising one or more first depolymerization products, and wherein the depolymerization conditions comprise a temperature of from 150° C. to 260° C. and a pressure of 10 atm to 70 atm; cooling at least a portion of the first mixture to a temperature of 150° C. or below; exposing the at least a portion of the first mixture to an alcohol composition and an alcoholysis catalyst under conditions including a temperature of from 23° C. to 90° C., and a pressure of from 1 atm to 2 atm for 0.5 hours to 5 hours to provide a second mixture, the second mixture comprising one or more dialkyl terephthalates; and separating at least a portion of the one or more dialkyl terephthalates by solid-liquid separation to provide a dialkyl terephthalate solid component and a filtrate.

2. The process of claim 1, wherein at least a portion of the methanol was recovered from a prior process of recovering dialkyl terephthalate from a prior polyester composition.

3. The process of claim 1, wherein the filtrate comprises one or more glycols and methanol.

4. The process of claim 3, further comprising: exposing a second polyester composition to at least a portion of the filtrate in the first reaction vessel under the depolymerization conditions to produce one or more second depolymerization products.

5. The process of claim 1, wherein a weight ratio of the amount of the one or more glycols relative to the amount of the methanol in the first reaction vessel is 1:20 to 100:1.

6. The process of claim 1, wherein the cooling at least a portion of the first mixture to a temperature of 150° C. or below comprises, cooling the at least a portion of the first mixture to a temperature of from 50° C. to 150° C.

7. The process of claim 1, further comprising: separating at least a portion of the one or more first depolymerization products in the first mixture from an insoluble component in the first mixture, wherein the separating occurs at a temperature of from 50° C. to 150° C.

8. The process of claim 1, wherein, during the exposing a first polyester composition to a first composition comprising one or more glycols and methanol in a first reaction vessel under depolymerization conditions, a weight ratio of the one or more glycols to the first polyester composition is in a range of about 1:9 to about 9:1.

9. The process of claim 1, wherein the one or more glycols comprises ethylene glycol (EG), diethylene glycol (DEG), triethylene glycol (TEG), 1,4-cyclohexanedimethanol (CHDM), poly(ethylene glycol) (PEG), neopentyl glycol (NPG), propane diol (PDO), butanediol (BDO), 2-methyl-2,4-pentanediol (MP diol), poly(tetramethylene ether)glycol (PTMG), or a combination thereof.

10. The process of claim 1, wherein a weight ratio of the alcohol composition to the first polyester composition can be in a range of about 2:1 to about 10:1.

11. The process of claim 1, wherein the first polyester composition comprises polyethylene terephthalate (PET), 1,4-cyclohexanedimethanol (CHDM)-modified PET, isophthalic acid (IPA)—modified PET, diethylene glycol (DEG)-modified PET, neopentyl glycol (NPG)-modified PET, propane diol (PDO)-modified PET, butanediol (BDO)-modified PET, heaxanediol (HDO)-modified PET, 2-methyl-2,4-pentanediol (MP diol)-modified PET, isosorbide-modified PET, poly(tetramethylene ether) glycol (PTMG)-modified PET, poly(ethylene glycol) (PEG)-modified PET, polycyclohexylenedimethylene terephthalate (PCT), cyclohexanedimethanol (CHDM)-containing copolyester, isosorbide-containing copolyester, or a combination thereof.

12. The process of claim 1, wherein the first polyester composition contains 0 mole % to 100 mole % CHDM, 0 mole % to 100 mole % DEG, 0 mole % to 100 mole % NPG, 0 mole % to 100 mole % PDO, 0 mole % to 100 mole % BDO, 0 mole % to 100 mole % HDO, 0 mole % to 100 mole % MP diol, 0 mole % to 100 mole % isosorbide, 0 mole % to 100 mole % PTMG, 0 mole % to 100 mole % PEG, and 0 mole % to 30 mole % isophthalic acid, wherein the sum of diol equivalents in the one or more polyesters is about 100 mole %, and wherein the sum of diacid equivalents in the first polyester composition is about 100 mole %.

13. The process of claim 1, wherein the polyester composition has an inherent viscosity of from about 0.1 dL/g to about 1.2 dL/g, as determined according to ASTM D2857-70.

14. The process of claim 1, wherein the first polyester composition comprises one or more foreign materials, wherein the one or more foreign materials are present in the polyester composition in an amount of from 0.01 wt. % to 50 wt. %, relative to the weight of one or more polyesters in the polyester composition, and wherein the one or more foreign materials comprise at least one member selected from the group consisting of polyesters other than polyethylene terephthalate, polyvinyl chloride (PVC), polyvinyl acetal, polyvinyl butyral (PVB), polyvinyl alcohol (PVOH), ethylene vinyl alcohol (EVOH), cotton, polyolefins, polyethylene, polypropylene, polystyrene, polycarbonate, Spandex, natural fibers, cellulose ester, polyacrylates, polymethacrylate, polyamides, nylon, poly(lactic acid), polydimethylsiloxane, polysilane, calcium carbonate, titanium dioxide, inorganic fillers, dyes, pigments, color toners, colorants, plasticizers, adhesives, flame retardants, metals, aluminum, and iron.

15. The process of claim 1, wherein the dialkyl terephthalate solid component comprises dimethyl terephthalate (DMT), and wherein the DMT is at least 90% pure.

16. The process of claim 1, wherein the exposing a first polyester composition to a first composition comprising one or more glycols and methanol in a first reaction vessel under depolymerization conditions comprises further exposing the first polyester composition to one or more glycolysis catalysts, wherein the one or more glycolysis catalysts comprise a member selected from the group consisting of Li₂CO₃, K₂CO₃, CaCO₃, Na₂CO₃, Cs₂CO₃, ZrCO₃, LiOH, NaOH, KOH, tetrabutylammonium hydroxide (TBAH), sodium methoxide (NaOMe), lithium methoxide (LiOMe), magnesium methoxide (Mg(OMe)₂, potassium t-butoxide, ethylene glycol monosodium salt, ethylene glycol disodium salt, tetra isopropyl titanate (TIPT), butyltin tris-2-ethylhexanoate (FASCAT 4102), 1,8-Diazabicyclo[5.4.0]undec-7-ene (DBU), zinc acetylacetonate hydrate (Zn(acac)₂), zinc acetate (Zn(OAc)₂), manganese (II) acetate (Mₙ(OAc)₂), hydrotalcite, zeolite, and lithium chloride.

17. The process of claim 14, further comprising: prior to the exposing the at least a portion of the first mixture to an alcohol composition and an alcoholysis catalyst, separating an insoluble component in the first mixture, wherein the insoluble component comprises the one or more foreign materials.

18. The process of claim 1, wherein the alcohol composition comprises methanol.

19. The process of claim 1, wherein, the alcoholysis catalyst is present in an amount of from 0.1 wt. % to 20 wt. %, relative to the weight of the first polyester composition, and wherein the alcoholysis catalyst comprises K₂CO₃, Na₂CO₃, Li₂CO₃, Cs₂CO₃; KOH, LiOH, NaOH; NaOMe, Mg(OMe)₂, KOMe, KOt-Bu, ethylene glycol monosodium salt, ethylene glycol disodium salt, or a combination thereof.

20. The process of claim 1, wherein the one or more depolymerization products comprise monomers, oligomers, or a combination thereof, and wherein the oligomers exhibit a degree of polymerization of from 2 to 10.
